(19) United States
(12) Patent Application Publication
(10) Pub. No.: US 2025/0134183 A1
(43) Pub. Date: May 1, 2025

(54) FORMULATIONS OF DEODORIZING AND ANTI-IRRITATING COATINGS FOR BRASSIERE PRODUCTS

(71) Applicant: Hung Hon Industrial Company Limited, Hong Kong (HK)

(72) Inventors: Wing Nien Wylie O, Hong Kong (HK); Chi Yin LEUNG, Hong Kong (HK); Caroline Wing In LAI, Hong Kong (HK); Sui Lung CHEUNG, Hong Kong (HK)

(21) Appl. No.: 18/690,266

(22) PCT Filed: Sep. 8, 2022
(86) PCT No.: PCT/CN2022/117812

§ 371 Date: Mar. 8, 2024

Related U.S. Application Data
(60) Provisional application No. 63/241,545, filed on Sep. 8, 2021.

(51) Int. Cl. A41C 3/12; A01N 41/04; A01P 1/00; A61L 2/232; A61L 101/48; A61L 101/50; D06B 3/18; D06M 13/00; D06M 13/272; D06M 13/41; D06M 13/46; D06M 15/03; D06M 16/00; D06M 101/32; D06M 101/34

(52) U.S. Cl. CPC A41C 3/12; A01N 41/04; A01P 1/00; A61L 2/232; D06B 3/18; D06M 13/005; D06M 13/272; D06M 13/41; D06M 13/46; D06M 15/03; D06M 16/00; A61L 2101/48; A61L 2101/50; A61L 2202/26; D06M 2101/32; D06M 2101/34

(57) ABSTRACT

A deodorizing and anti-irritating brassiere is presented. The brassiere includes one or more fabric portions and the fabric portions are made of nylon, polyester, cotton, and polyurethane (e.g., Spandex); these include fabric blends of nylon, polyester, cotton, and polyurethane/Spandex. The brassiere has a deodorizing and anti-irritating coating that includes a deodorizing, bactericidal and fungicidal component of

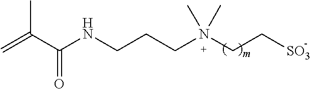

wherein m is 2, 3, 4, 5, 6, 7, 8, 9 and a deodorizing component that includes the following compound:

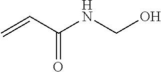

and a compound according to the following formula (I)

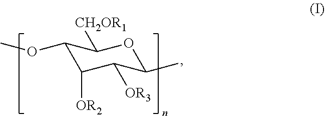

wherein n is 6, 7, 8; R₁, R₂, and R₃ are jointly or independently selected from H or

CH₂CH₂SO₃⁻Na⁺ (II)

wherein the total number of groups represented by formula (II) can be 2, 4.

### CROSS-REFERENCE TO RELATED APPLICATIONS

The present application claims priority to U.S. provisional patent application 63/241,545 filed 8 Sep. 2021, the disclosure of which is incorporated by reference herein.

### FIELD OF THE INVENTION

The present invention relates to the field of coated fabrics and, more particularly, to brassieres coated with deodorizing, antibacterial, and anti-irritating compositions.

### BACKGROUND

Brassiere products are intimate apparel that have direct contact with the skin. Malodor usually provide unpleasant experience to users of brassiere products, and users with sensitive skin are vulnerable to organic volatiles and sweat. Body malodor from sweat is a source of malodor. Users with sensitive skin are particularly vulnerable to emitted organic compounds and to body sweat. Bromhidrosis is a condition related to body malodor, and is usually related to sweat secretions by apocrine glands including those in the breast areas. Bacteria on the body, such as Staphylococcus hominis, metabolize amino acids to generate ammonia, and odorous fatty acids of short carbon chains such as propionic acid and isovaleric acid. Further, Staphylococcus hominis is also responsible for breaking down sweat into sulfhydryl compounds. The combinations of ammonia, isovaleric acid and sulfhydryl compounds are the main contributors to body malodor, and can create hygiene problems for women. Further, aerobic microorganisms that are present in apocrine glands, such as Corynebacterium, metabolize compounds such as L-cysteine in sweat into odorous sulfhydryl compounds.

Another source of malodor are volatiles that arise from foam pad manufacturing. These involve residual volatile organic compounds (VOCs) such as benzene, toluene and styrene from the manufacturing and foaming process of the polyurethane foam of brassiere pads.

Brassiere product users who are breastfeeding mothers are often concerned about the stagnant smell of breast milk residue on the fabric of brassiere products. Formation of these odors evolves via hydroperoxide generation from the respective polyunsaturated fatty acids (PUFAs) in human milk. Oxidation can take place via enzymatically catalyzed processes or via autoxidation mechanisms. Compounds including but not limited to the mushroom-like smelling 1-octen-3-one, the metallic and blood-like smelling compounds trans-4,5-epoxy-(E)-2-decenal and the musty-smelling compounds decanoic and dodecanoic acid were detected in the stored and denatured breast milk sample. These usually contribute to a stagnant smell.

Prolonged usage of brassiere products will trap sweat that can induce bacterial and fungal infections and irritation of skin. Users might suffer from ‘heat rash’, which happens when sweat is trapped due to blockage in sweat glands in the deeper layers of the skin, causing a sensation of irritation and sensitized skin. Sensitized skin is prone to Staphylococcus infections or fungal infections caused by fungus, such as Malassezia, which can further block sweat glands and create skin infection problems.

Commercial fabric coating treatments that combat bacteria and sweat odors are available. They are, however, only specific towards carboxylic acids in body odor, not other components such as sulfhydryl compounds, toluene and styrene. Further, active ingredients in commercial coatings typically leach up to 70% when the fabric products are washed as few as 10 times.

There is a need for deodorizing and anti-irritating coatings applicable to fabric for brassiere applications that can provide extra benefits of comfort and hygiene to users, including people with sensitive skin and breastfeeding mothers, improving quality of women's lives. The present invention addresses this need.

### SUMMARY OF THE INVENTION

The present invention provides a deodorizing and anti-irritating coating applicable to fabrics for brassiere applications. The coating acts as a barrier on the product to provide deodorizing performance against volatile compounds, malodors from denatured breast milk and from body sweat. The coating is non-irritating to users and durable when washed in domestic laundry equipment. In examples, the polymer components of the coating exhibit strong adhesion to fabric surfaces such as fabrics that are up to 100% polyester, up to 100% nylon, and at least 85% cotton. The coatings are able to withstand lamination and pressing conditions onto polyurethane foam during manufacturing. Importantly, the coated brassieres do not include silver/silver nanoparticles which both increase costs and may result in skin irritation.

In one aspect, the present invention provides a deodorizing, anti-irritating, bactericidal, and launderable brassiere with a coating formed thereon. In another aspect, the present invention provides a coating composition for providing the coating layer. The brassiere includes one or more fabric portions that include one or more of nylon, polyester, cotton, and polyurethane, including fabric blends of one or more of nylon, polyester, cotton, and polyurethane. The brassiere has a deodorizing and anti-irritating coating formed thereon that include a deodorizing, bactericidal and fungicidal component of:

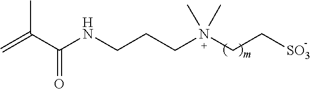

wherein m is 2, 3, 4, 5, 6, 7, 8, 9 and a deodorizing component that includes the following compound:

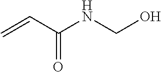

and a cyclodextrin compound according to the following formula (I)

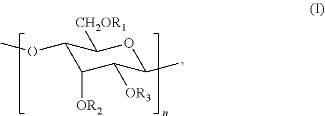

wherein n is 6, 7, 8; R₁, R₂, and R₃ are jointly or independently selected from H or

CH₂CH₂SO₃⁻Na⁺ (II)

wherein the total number of groups represented by formula (II) can be 2, 4,
and one or more crosslinkers, one or more initiators; and/or one or more additives.

In a further aspect, a ratio of

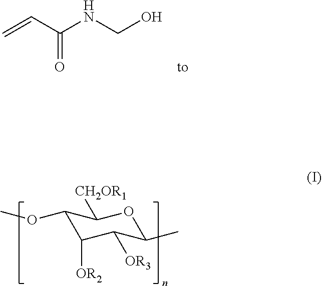

is 0.7:1 to 1.6:1 by weight.

In a further aspect, the compound of formula I includes

CH₂CH₂SO₃⁻Na⁺ (II)

and a total number of

CH₂CH₂SO₃⁻Na⁺ (II)

is 2.

In a further aspect, the compound of formula I includes

CH₂CH₂SO₃⁻Na⁺ (II)

and a total number of

CH₂CH₂SO₃⁻Na⁺ (II)

is 4.

In a further aspect, a ratio of

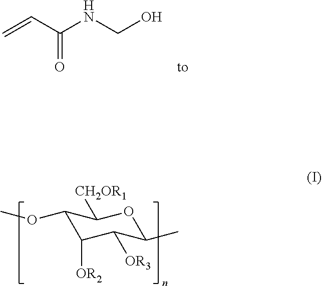

is 1.3:1 by weight.

In a further aspect, the initiator is selected from one or more of 2,2′-azobis [2-(2-imidazolin-2-yl)propane] disulfate dihydrate, 2,2′-azobis [2-(2-imidazolin-2-yl)propane] dihydrochloride, 2,2′-azobis (2-methylpropionamidine) dihydrochloride, 2,2′-azobis [2-(2-imidazolin-2-yl)propane], 2,2′-azobis [n-(2-carboxyethyl)-2-methylpropionamidine] n-hydrate, 2,2′-azobis [2-methyl-n-(2-hydroxyethyl) propionamide], or 4,4′-azobis (4-cyanovaleric acid).

In a further aspect, the one or more additives are selected from acrylic acid, 2-(dimethylamino) ethyl acrylate, 3-(trimethoxysilyl) propyl acrylate, glycidyl acrylate, methacrylic acid, 2-acrylamido-2-methylpropanesulfonic acid, N-[3-(dimethylamino)-propyl] acrylamide, N-[2-(dimethylamino)-ethyl] acrylamide, N-(3-dimethylaminopropyl)-methacrylamide.

In a further aspect, the coating includes a metal salt. The metal salt may be one or more of zinc acrylate, sodium acrylate, potassium acrylate, zinc methacrylate, 2-acrylamido-2-methylpropanesulfonate sodium salt or 3-sulfopropyl acrylate potassium salt.

### DETAILED DESCRIPTION

In one aspect, the present invention provides a deodorizing and anti-irritating brassiere. The brassiere includes one or more fabric portions and the fabric portions are made of nylon, polyester, cotton, and polyurethane (e.g., Spandex); these include fabric blends of nylon, polyester, cotton, and polyurethane/Spandex. The brassiere has a deodorizing and anti-irritating coating that includes a deodorizing, bactericidal and fungicidal component of

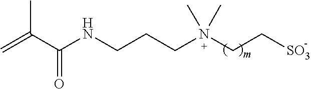

wherein m is 2, 3, 4, 5, 6, 7, 8, 9 and a deodorizing component that includes the following compound:

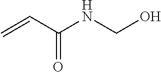

and a cyclodextrin compound according to the following formula (I)

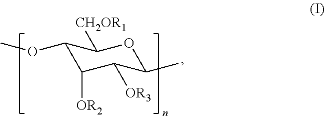

wherein n is 6, 7, 8; R1, R2, and R3 are jointly or independently selected from H or

CH₂CH₂SO₃⁻Na⁺ (II)

wherein the total number of groups represented by formula (II) can be 2, 4
wherein a ratio of

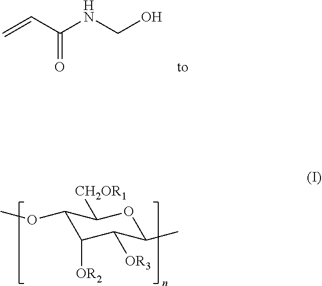

is 0.7:1 to 1.6:1 by weight.

The deodorizing, bactericidal and fungicidal component of

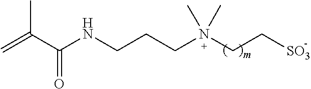

wherein m is 2 is 3- [(3-methacrylamidopropyl) dimethylammonio] propane-1-sulfonate and is capable to demonstrate excellent deodorizing, bactericidal and fungicidal properties. The positive charge of zwitterion shall be able to neutralize basic odorous molecules such as ammonia. The negative charge of zwitterion is able to neutralize acidic odorous molecules including isovaleric acid and methyl mercaptan via the sulfonate groups. In addition, the group exhibits bactericidal and fungicidal properties by allowing destruction of cell membranes via physical cell lysis or charge disruption of bacteria and fungi (cell lysis).

Formula (I) where n is 7 represents a beta-cyclodextrin. The functional groups of formula (II) that may be used as R₁, R₂, and/or R₃ form sulfonated beta-cyclodextrin. Sulfonated beta-cyclodextrin is capable to neutralize odorous compounds with acidic moieties, such as isovaleric acid and methyl mercaptan. The hydrophobic cavity of cyclodextrin allows capturing of neutral VOCs, such as toluene and nonenal.

Use of cyclodextrins is advantageous as they may block pore-forming toxins and utilized as inhibitors of toxins and anti-infectives. In particular molecules having the same symmetry as the target pores are highly potent inhibitors.

As will be discussed in further detail below in the Examples, the coating layer may be provided to the brassieres through a simple dip-coating process. The dip coating solutions used to form the coating layer may include various additional components including, for example, crosslinkers, initiators, additives, and metal salts.

Co-polymerization of suitable components provides a deodorizing and anti-irritating coating when applied on fabric that is durable against home laundry, and is heat resistant during the brassiere pad lamination process. The deodorizing, bactericidal and fungicidal component of

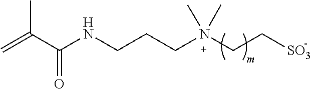

wherein m is 2, 3, 4, 5, 6, 7, 8, 9 and a deodorizing component that includes the following compound:

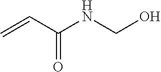

and a cyclodextrin compound according to the following formula (I)

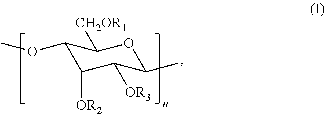

wherein n is 6, 7, 8; R1, R2, and R3 are jointly or independently selected from H or

CH₂CH₂SO₃⁻Na⁺ (II)

wherein the total number of groups represented by formula (II) can be 2, 4
wherein a ratio of

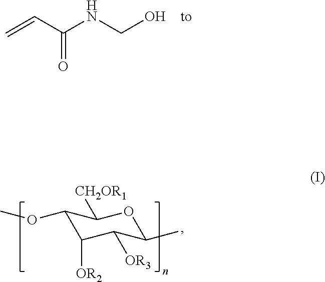

is 0.7:1 to 1.6:1 by weight
of the deodorizing and anti-irritating coating on brassieres are linked together via co-polymerization during the dip-coating process. The acrylamide groups of the bactericidal and fungicidal component of component of

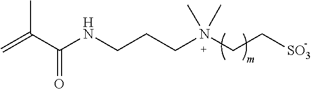

wherein m is 2, 3, 4, 5, 6, 7, 8, 9 and the compound of deodorizing component:

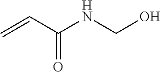

that is installed on the other compound according to the following formula (I)

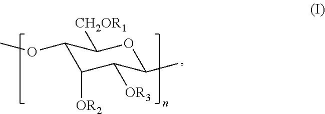

wherein n is 6, 7, 8; R1, R2, and R3 are jointly or independently selected from H or

CH₂CH₂SO₃⁻Na⁺ (II)

wherein the total number of groups represented by formula (II) can be 2, 4 wherein a ratio of

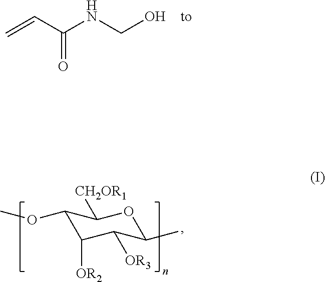

is 0.7:1 to 1.6:1 by weight enable co-polymerization to take place in water during the dip-coating process for the formation of the said coating when applied on fabric surface. Crosslinkers that comprise acrylamide groups further enable co-polymerization to take place in water during the dip-coating process for the formation of the said coating when applied on fabric surface. Examples of crosslinkers include one or more of N,N′-methylenebismethacrylamide, N,N′-methylenebismethacrylamide, N,N′-methylenebisacrylamide, N,N′-bis (2-acrylamidoethyl)acrylamide, N-[tris (3-acrylamidopropoxymethyl) methyl] acrylamide, N,N′-[oxybis (2,1-ethanediyloxy-3,1-propanediyl)] bisacrylamide, N,N′-1,2-ethanediylbis {n-[2-(acryloylamino) ethyl] acrylamide}, diethylene glycol dimethacrylate, diethylene glycol diacrylate, or one or more of the following compounds:

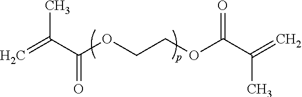

where p is 4 to 14, or

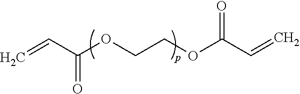

where q is 4 to 14.

The dip coating solution may further include one or more initiators. The initiator(s) are selected from compounds that can react with the acrylamide groups of the bactericidal and fungicidal component of component of

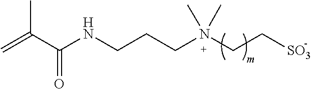

wherein m is 2, 3, 4, 5, 6, 7, 8, 9 and the compound of deodorizing component:

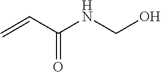

that is installed on the other compound according to the following formula (I)

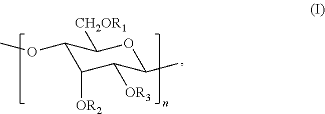

wherein n is 6, 7, 8; R1, R2, and R3 are jointly or independently selected from H or

CH₂CH₂SO₃⁻Na⁺ (II)

wherein the total number of groups represented by formula (II) can be 2, 4 wherein a ratio of

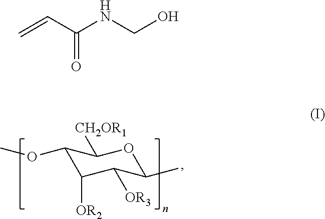

is 0.7:1 to 1.6:1 by weight
to enable co-polymerization to take place in water during the dip-coating process for the formation of the said coating when applied on fabric surface. The initiator(s) may be one or more of 2,2′-azobis [2-(2-imidazolin-2-yl) propane] disulfate dihydrate, 2,2′-azobis [2-(2-imidazolin-2-yl)propane] dihydrochloride, 2,2′-azobis (2-methylpropionamidine) dihydrochloride, 2,2′-azobis [2-(2-imidazolin-2-yl) propane], 2,2′-azobis [n-(2-carboxyethyl)-2-methylpropionamidine] n-hydrate, 2,2′-azobis [2-methyl-n-(2-hydroxyethyl)propionamide], or 4,4′-azobis (4-cyanovaleric acid).

Additional additives may be present in the dip-coating solution to enhance rheological properties of the solution or to enhance adhesion to the fabric. Such additives include one or more of acrylic acid, 2-(dimethylamino) ethyl acrylate, 3-(trimethoxysilyl) propyl acrylate, glycidyl acrylate, methacrylic acid, 2-acrylamido-2-methylpropanesulfonic acid, N-[3-(dimethylamino)-propyl] acrylamide, N-[2-(dimethylamino)-ethyl] acrylamide, N-(3-dimethylaminopropyl)-methacrylamide.

The metal salts used in the dip coating solution may be one or more of zinc acrylate, sodium acrylate, potassium acrylate, zinc methacrylate, 2-acrylamido-2-methylpropanesulfonate sodium salt, and/or 3-sulfopropyl acrylate potassium salt. The metal salts may bind to the deodorizing, bactericidal and fungicidal component of

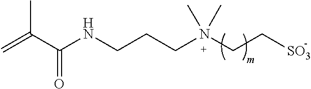

wherein m is 2, 3, 4, 5, 6, 7, 8, 9
The bound metal ions are capable of inducing an oligodynamic effect on incoming bacteria at low concentrations. One mechanism involves the oxidation of metal ions; as the ions oxidize, they use electrons from bacterial cell membranes, destroying the membrane. Alternatively, uptake of metal ions in large quantities by bacteria create a toxic condition and the bacteria are killed.

It is noted that metal salts set forth above do not require the presence of silver. That is, the present invention provides a silver-free fabric treatment system; silver is not required to provide the bactericidal properties of the treated fabric.

The components of the solution described above may be easily applied to fabric by a dip-coating technique. The term “dip-coat,” as used herein, broadly relates to any technique in which a fabric is immersed in a solution with or without mechanical agitation during the dip-coating.

A variety of fabrics may use the fabric finish of the present invention. Such fabrics include, but are not limited to polyester-based fabrics, nylon-based fabrics, urethane-based fabrics (e.g., spandex), cotton-based fabrics.

The dip-coating may be performed at room temperature (defined as approximately 20-27° C.) or it may occur at an elevated temperature of 28-80°° C. The duration of the dip-coating may range from approximately 5 to 60 minutes depending in part upon the weight and density of the fabric. Following dip-coating the fabric may optionally be washed with water, followed by drying/curing. Drying may be by air-drying or drying at elevated temperature. The drying time will depend upon the weight of the fabric but typically the time is about 5 to 60 minutes at a temperature of about 60 to 180° C.

A brassiere having the launderable bactericidal, deodorizing, and anti-irritation fabric finish prepared as described above has bactericidal activity of at least 90% in terms of reducing bacterial growth and activity from bacteria including one or more of Staphylococcus aureus, Klebsiella pneumonia, and has deodorizing performance of at least 90% including one of more of acetic acid, isovaleric acid and ammonia. Details of fabric testing against bacteria are set forth in the Examples, below. The coating weight of the fabric finish ranges from approximately 10.3 to approximately 70.9 g/m².

### EXAMPLES

### Example 1

Table 1 below provides a solution for dip-coating a polyester fabric in order to form a deodorizing and anti-irritating fabric coating in said polyester fabric.

TABLE 1

| | Amount |
|---|---|
| | (weight/ |
| Components | volume %) |
| 3-[(3-methacrylamidopropyl)dimethylammonio]propane- | 5 |
| 1-sulfonate | |
| Deodorizing component | 3.6 |
| N-[3-(dimethylamino)propyl]methacrylamide | 0.7 |
| 2-acrylamido-2-methyl-1-propanesulfonic acid | 0.9 |
| 2,2′-azobis(2-methylpropionamidine) dihydrochloride | 0.05 |
| Distilled water | 100 |

Deodorizing component includes the following compound:

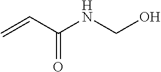

and a compound according to the following formula (I)

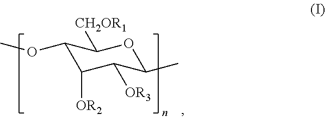

wherein n is 7; R₁, R₂, and R₃ are jointly or independently selected from H or

CH₂CH₂SO₃⁻Na⁺ (II)

wherein the total number of groups represented by formula (II) is 2
wherein a ratio of

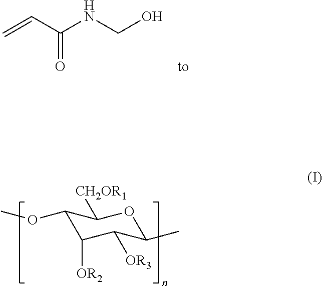

is 1.3 to 1 by weight.

### Dip-Coating Procedure:

All the components in Table 1 were dissolved in distilled water at 25 degrees Celsius to form the coating solution.

100% Polyester fabric was soaked in the above coating solution at liquid ratio of 1:16.5 for 5 minutes at 25 degrees Celsius with stirring.

The fabric was then padded.

This fabric was then dried and cured at 160 degrees Celsius for 9 minutes and 25 seconds.

The coated fabric was washed with distilled water for 25 minutes to remove water-soluble residual materials, and tumble dried at 80 degrees Celsius for 30 minutes.

The coating weighed from 24.4 to 26.6 g/m2 upon applying the finish on the dip-coated fabric after drying.

Table 2 below summarizes the deodorizing performance of Example 1.

TABLE 2

| Test | | Deodorizing | |
|---|---|---|---|
| Article | Malodor | Performance (%) | Test Method |
| Example 1 | Isovaleric acid | 98 | ISO 17299-3 |

### Example 2

Table 3 below provides a solution for dip-coating a polyester fabric in order to form a deodorizing and anti-irritating fabric coating in said polyester fabric.

TABLE 3

| | Amount |
|---|---|
| Components | (weight/volume %) |
| 3-[(3-methacrylamidopropyl)dimethylammonio] | 5.0-9.9 |
| propane-1-sulfonate | |
| Deodorizing component | 3.6-7.2 |
| 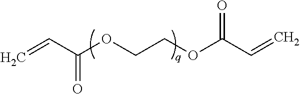 | 1.1-2.2 |
| where q is 9 | |
| N-[3-(dimethylamino)propyl]methacrylamide | 0.7-1.4 |
| 2-acrylamido-2-methyl-1-propanesulfonic acid | 0.9-1.8 |
| 2,2′-azobis(2-methylpropionamidine)dihydrochloride | 0.05-0.09 |
| distilled water | 100 |

Deodorizing component includes the following compound:

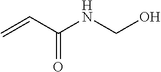

and a compound according to the following formula (I)

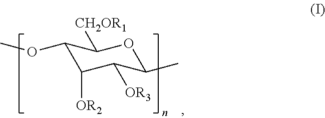

wherein n is 7; R₁, R₂, and R₃ are jointly or independently selected from H or

CH₂CH₂SO₃⁻Na⁺ (II)

wherein the total number of groups represented by formula (II) is 2
wherein a ratio of

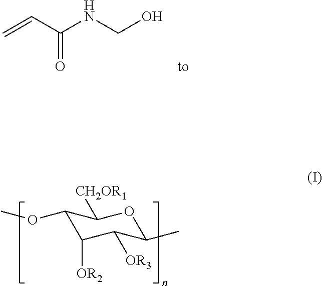

is 1.3 to 1 by weight.

### Dip-Coating Procedure:

All the components in Table 3 were dissolved in distilled water at 25 degrees Celsius to form the coating solution.

100% Polyester fabric was soaked in the above coating solution at liquid ratio of 1:16.5 for 5 minutes at 25 degrees Celsius with stirring.

The fabric was then padded.

This fabric was then dried and cured at 160 degrees Celsius for 9 minutes and 25 seconds.

The coated fabric was then washed with distilled water for 25 minutes to remove water-soluble residual materials, and tumble dried at 80 degrees Celsius for 30 minutes.

A coating weighing from 28.3 to 70.9 g/m² resulted upon applying the finish to fabric after drying.

Table 4 below summarizes the deodorizing performance of Example 2.

TABLE 4

| Test | | Deodorizing | |
|---|---|---|---|
| Article | Malodor | Performance (%) | Test Method |
| Example 2 | Isovaleric acid | 95-96 | ISO 17299-3 |

### Example 3

Table 5 below provides a solution for dip-coating a polyester fabric in order to form a deodorizing and anti-irritating fabric coating in said polyester fabric.

TABLE 5

| | Amount |
|---|---|
| Components | (weight/volume %) |
| 3-[(3-methacrylamidopropyl)dimethylammonio] | 5.0 |
| propane-1-sulfonate | |
| Deodorizing component | 3.6 |
| 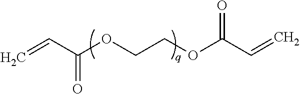 | 1.1 |
| where q is 9 | |
| N-[3-(dimethylamino)propyl]methacrylamide | 0.7 |
| 2-acrylamido-2-methyl-1-propanesulfonic acid | 0.9 |
| 2,2′-azobis(2-methylpropionamidine)dihydrochloride | 0.05 |
| distilled water | 100 |

Deodorizing component includes the following compound:

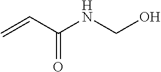

and a compound according to the following formula (I)

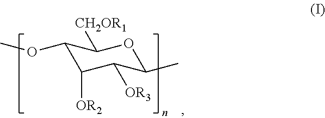

wherein n is 7; R₁, R₂, and R₃ are jointly or independently selected from H or

CH₂CH₂SO₃⁻Na⁺ (II)

wherein the total number of groups represented by formula (II) is 2,
wherein a ratio of

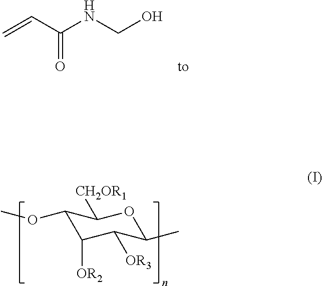

is 1.3 to 1 by weight.

### Dip-Coating Procedure:

All the components in Table 5 were dissolved in distilled water at 25 degrees Celsius to form the coating solution.

100% Polyester fabric was dip-coated with the above coating solution at a liquid ratio of 1:16.5 at 25 degrees Celsius for 5 minutes.

The fabric was then padded.

This fabric was then dried and cured at 180 degrees Celsius for 5 minutes and 6 seconds.

The coated fabric was then washed with distilled water for 25 minutes to remove water-soluble residual materials, and tumble dried at 80 degrees Celsius for 30 minutes.

A coating weighing from 26.9 to 30.8 g/m² resulted upon applying the finish to fabric after drying.

Table 6 below summarizes bactericidal activity of Example 3:

TABLE 6

| | | | Bactericidal | Bactericidal Activity | |
|---|---|---|---|---|---|
| Test | Bacteria | Contact | Activity | after 30 Laundry | Test |
| Article | Strains | Time (h) | (%) | Cycles (%)* | Method |
| Example 3 | Staphylococcus | 24 | 99.9 | 99.9 | AATCC |
| | aureus | | | | 100-2019 |
| | Klbesiella | | 99.9 | 99.9 | |
| | pneumonia | | | | |

*Washed using methods of AATCC LP1 - 30 cycles of 12 minutes machine wash at 105° F., normal cycle washing with laundry bag, tumble dry delicate

Table 7 below summarizes the deodorizing performance of Example 3

TABLE 7

| | | | Deodorizing | |
|---|---|---|---|---|
| | | Deodorizing | Performance (%) | |
| Test | | Performance | after 30 Laundry | |
| Article | Malodor | (%) | Cycles (%)* | Test Method |
| Example 3 | Ammonia | 89 | 51 | ISO 17299-2 |
| | Isovaleric | 96 | 91 | ISO 17299-3 |
| | acid | | | |

*Washed using methods of AATCC LP1 - 30 cycles of 12 minutes machine wash at 105° F., normal cycle washing with laundry bag, tumble dry delicate

### Example 4

Table 8 below provides a solution for dip-coating a polyester fabric in order to form a deodorizing and anti-irritating fabric coating on the polyester fabric.

TABLE 8

| | Amount |
|---|---|
| Components | (weight/volume %) |
| 3-[(3-methacrylamidopropyl)dimethylammonio] | 5.3 |
| propane-1-sulfonate | |
| Deodorizing component | 4.2 |
| 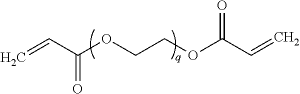 | 1.2 |
| where q is 9 | |
| N-[3-(dimethylamino)propyl]methacrylamide | 0.8 |
| 2-acrylamido-2-methyl-1-propanesulfonic acid | 0.9 |
| 2,2′-azobis(2-methylpropionamidine)dihydrochloride | 0.05 |
| zinc acrylate | 0.9 |
| distilled water | 100 |

Deodorizing component includes the following compound:

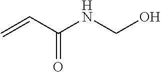

and a compound according to the following formula (I)

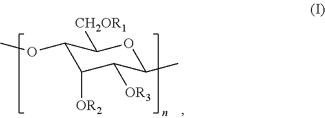

wherein n is 7; R₁, R₂, and R₃ are jointly or independently selected from H or

CH₂CH₂SO₃⁻Na⁺ (II)

wherein the total number of groups represented by formula (II) is 4
wherein a ratio of

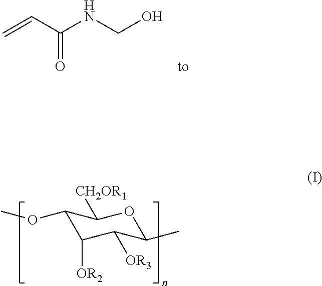

is 1.3 to 1 by weight.

### Dip-Coating Procedure:

All the components in Table 8 were added to distilled water at 25 degrees Celsius to form a suspension.

The above suspension was filtered by gravity to form the coating solution.

100% Polyester fabric was dip-coated with the above coating solution at a liquid ratio of 1:15.5 for at 25 degrees Celsius for 5 minutes.

The fabric was then padded.

This fabric was then dried and cured at 175 degrees Celsius for 3 minutes and 5 seconds.

The coated fabric was then washed with distilled water for 25 minutes to remove water-soluble residual materials, and tumble dried at 80 degrees Celsius for 30 minutes.

A coating weighing from 14.7 to 15.4 g/m² resulted upon applying the finish to fabric after drying.

Table 9 below summarizes the deodorizing performance of Example 4

TABLE 9

| | | | Deodorizing | |
|---|---|---|---|---|
| | | Deodorizing | Performance (%) | |
| Test | | Performance | after 30 Laundry | |
| Article | Malodor | (%) | Cycles (%)* | Test Method |
| Example 4 | Ammonia | 94 | 53 | ISO 17299-2 |

*Washed using methods of AATCC LP1 - 30 cycles of 12 minutes machine wash at 105° F., normal cycle washing with laundry bag, tumble dry delicate.

### Example 5

Table 10 below provides a solution for dip-coating a polyester fabric in order to form a deodorizing and anti-irritating fabric coating on the polyester fabric.

TABLE 10

| | Amount |
|---|---|
| Components | (weight/volume %) |
| 3-[(3-methacrylamidopropyl)dimethylammonio] | 5.3 |
| propane-1-sulfonate | |
| Deodorizing component | 4.2 |
| 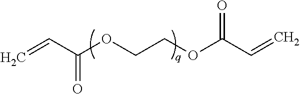 | 1.2 |
| where q is 9 | |
| N-[3-(dimethylamino)propyl]methacrylamide | 0.8 |
| 2-acrylamido-2-methyl-1-propanesulfonic acid | 0.9 |
| acrylic acid | 0.3 |
| 2,2′-azobis(2-methylpropionamidine)dihydrochloride | 0.05 |
| distilled water | 100 |

Deodorizing component includes the following compound:

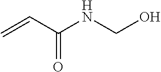

and a compound according to the following formula (I)

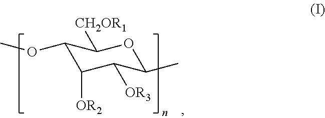

wherein n is 7; R₁, R₂, and R₃ are jointly or independently selected from H or

CH₂CH₂SO₃⁻Na⁺ (II)

wherein the total number of groups represented by formula (II) is 4
wherein a ratio of

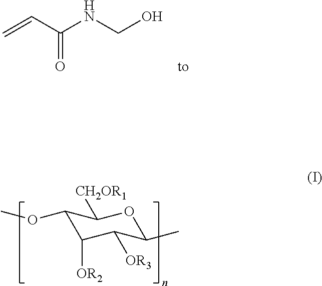

is 1.3 to 1 by weight.

### Dip-Coating Procedure:

All the components in Table 10 were added to distilled water at 25 degrees Celsius to form a suspension.

The above suspension was filtered by gravity to form the coating solution.

100% Polyester fabric was dip-coated with the above coating solution at a liquid ratio of 1:15.5 for at 25 degrees Celsius for 5 minutes.

The fabric was then padded.

This fabric was then dried and cured at 175 degrees Celsius for 3 minutes and 5 seconds.

The coated fabric was then washed with distilled water for 25 minutes to remove water-soluble residual materials, and tumble dried at 80 degrees Celsius for 30 minutes.

A coating weighing from 19.1 g/m² resulted upon applying the finish to fabric after drying.

Table 11 below summarizes the deodorizing performance of Example 5

TABLE 11

| Test | | Deodorizing | |
|---|---|---|---|
| Article | Malodor | Performance (%) | Test Method |
| Example 5 | Ammonia | 94 | ISO 17299-2 |

### Example 6

Further to the fabric from Example 5, this fabric was dipped for 5 minutes at 25 degrees Celsius into a 5.0% zinc acetate solution dissolved in 184 mL of distilled water. This fabric was then dried at 120 degrees Celsius for 2 minutes, and the said fabric was then washed with distilled water for 25 minutes to remove water-soluble residual materials, and tumble dried at 80 degrees Celsius for 30 minutes. A coating was weighted 20.5 g/m² upon applying the finish on fabric after drying. This fabric is capable of deodorize ammonia at 93% per ISO 17299-2.

### Example 7

Table 12 below provides a solution for dip-coating a nylon fabric in order to form

a deodorizing and anti-irritating fabric coating on the nylon fabric.

TABLE 12

| | Amount |
|---|---|
| Components | (weight/volume %) |
| 3-[(3-methacrylamidopropyl)dimethylammonio] | 5.3 |
| propane-1-sulfonate | |
| Deodorizing component | 4.2 |
| 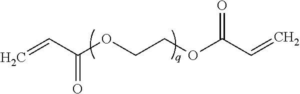 | 1.2 |
| where q is 9 | |
| N-[3-(dimethylamino)propyl]methacrylamide | 0.8 |
| 2-acrylamido-2-methyl-1-propanesulfonic acid | 0.9 |
| 2,2′-azobis(2-methylpropionamidine)dihydrochloride | 0.05 |
| zinc acrylate | 0.9 |
| distilled water | 100 |

Deodorizing component includes the following compound:

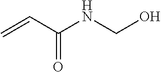

and a compound according to the following formula (I)

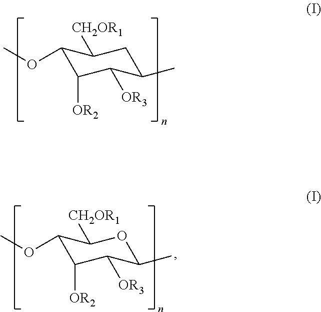

wherein n is 7; R₁, R₂, and R₃ are jointly or independently selected from H or

CH₂CH₂SO₃⁻Na⁺ (II)

wherein the total number of groups represented by formula (II) is 4
wherein a ratio of

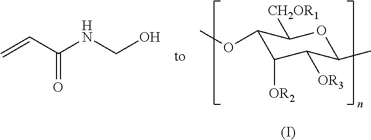

is 1.3 to 1 by weight.

### Dip-Coating Procedure:

All the components in Table 12 were added to distilled water at 25 degrees Celsius to form a suspension.

The above suspension was filtered by gravity to form the coating solution.

100% Nylon fabric was dip-coated with the above coating solution at a liquid ratio of 1:15.5 for at 25 degrees Celsius for 5 minutes.

The fabric was then padded.

This fabric was then dried and cured at 165 to 175 degrees Celsius for 4 minutes and 40 seconds.

The coated fabric was then washed with distilled water for 25 minutes to remove water-soluble residual materials, and tumble dried at 80 degrees Celsius for 30 minutes.

A coating weighing from 10.3 to 28.5 g/m² resulted upon applying the finish to fabric after drying.

Table 13 below summarizes the deodorizing performance of Example 7

TABLE 13

| Test | | Deodorizing | |
|---|---|---|---|
| Article | Malodor | Performance (%) | Test Method |
| Example 7 | Isovaleric Acid | 97-98 | ISO 17299-3 |

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of ±10%, ±5%, ±1%, or ±0.5% of the given value or range. The range may be indicated herein as from one endpoint to another endpoint or between two endpoints. Unless otherwise specified, all the ranges disclosed in the present disclosure include endpoints. When reference is made to “substantially” the same numerical value or characteristic, the term may refer to a value within ±10%, ±5%, ±1%, or ±0.5% of the average of the values.

Several embodiments of the present disclosure and features of details are briefly described above. The embodiments described in the present disclosure may be easily used as a basis for designing or modifying other processes and structures for realizing the same or similar objectives and/or obtaining the same or similar advantages introduced in the embodiments of the present disclosure. Such equivalent construction does not depart from the spirit and scope of the present disclosure, and various variations, replacements, and modifications can be made without departing from the spirit and scope of the present disclosure.

1. A deodorizing, anti-irritating, bactericidal, and launderable brassiere comprising:
a brassiere including one or more fabric portions, the fabric portions including one or more of nylon, polyester, cotton, and polyurethane, including fabric blends of one or more of nylon, polyester, cotton, and polyurethane; the brassiere having a deodorizing and anti-irritating coating formed thereon, the deodorizing and anti-irritating coating including:
a deodorizing, bactericidal and fungicidal component of 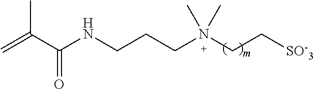
wherein m is 2, 3, 4, 5, 6, 7, 8, 9 and a deodorizing component that includes the following compound: 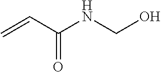
and a cyclodextrin compound according to the following formula (I) 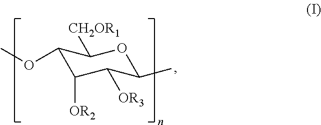
wherein n is 6, 7, 8; R₁, R₂, and R₃ are jointly or independently selected from H or
CH₂CH₂SO₃⁻Na⁺ (II)
wherein the total number of groups represented by formula (II) can be 2 or 4;
and
one or more crosslinkers;
and
one or more initiators; and/or
one or more additives.

2. The deodorizing, anti-irritating, bactericidal, and launderable brassiere according to claim 1, wherein a ratio of 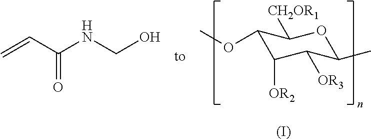
is 0.7:1 to 1.6:1 by weight.

3. The deodorizing, anti-irritating, bactericidal, and launderable brassiere according to claim 1, wherein the compound of formula I includes
CH₂CH₂SO₃⁻Na⁺ (II)
and a total number of
CH₂CH₂SO₃⁻Na⁺ (II)
is 2.

4. The deodorizing, anti-irritating, bactericidal, and launderable brassiere according to claim 1, wherein the compound of formula I includes
CH₂CH₂SO₃⁻Na⁺ (II)
and a total number of
CH₂CH₂SO₃⁻Na⁺ (II)
is 4.

5. The deodorizing, anti-irritating, bactericidal, and launderable brassiere according to claim 1, wherein the initiator is selected from one or more of 2,2′-azobis [2-(2-imidazolin-2-yl)propane] disulfate dihydrate, 2,2′-azobis [2-(2-imidazolin-2-yl) propane] dihydrochloride, 2,2′-azobis (2-methylpropionamidine) dihydrochloride, 2,2′-azobis [2-(2-imidazolin-2-yl)propane], 2,2′-azobis [n-(2-carboxyethyl)-2-methylpropionamidine] n-hydrate, 2,2′-azobis [2-methyl-n-(2-hydroxyethyl) propionamide], or 4,4′-azobis (4-cyanovaleric acid).

6. The deodorizing, anti-irritating, bactericidal, and launderable brassiere according to claim 1, wherein the one or more additives is selected from acrylic acid, 2-(dimethylamino) ethyl acrylate, 3-(trimethoxysilyl)propyl acrylate, glycidyl acrylate, methacrylic acid, 2-acrylamido-2-methylpropanesulfonic acid, N-[3-(dimethylamino)-propyl] acrylamide, N-[2-(dimethylamino)-ethyl] acrylamide, N-(3-dimethylaminopropyl)-methacrylamide.

7. The deodorizing, anti-irritating, bactericidal, and launderable brassiere according to claim 1, wherein the deodorizing and anti-irritating coating further comprises a metal salt.

8. The deodorizing, anti-irritating, bactericidal, and launderable brassiere according to claim 7, wherein the metal salt is selected from one or more of zinc acrylate, sodium acrylate, potassium acrylate, zinc methacrylate, 2-acrylamido-2-methylpropanesulfonate sodium salt or 3-sulfopropyl acrylate potassium salt.

9. A deodorizing, anti-irritating, bactericidal, and launderable coating solution for brassieres, comprising:
a deodorizing, bactericidal and fungicidal component of 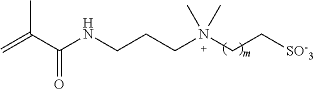
wherein m is 2, 3, 4, 5, 6, 7, 8, 9 and a deodorizing component that includes the following compound: 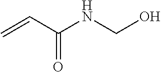
and a compound according to the following formula (I) 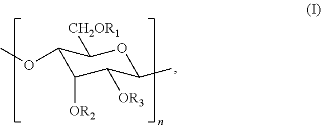
wherein n is 6, 7, 8; R₁, R₂, and R₃ are jointly or independently selected from H or
CH₂CH₂SO₃⁻Na⁺ (II)
wherein the total number of groups represented by formula (II) can be 2 or 4;
and
one or more crosslinkers;
and
one or more initiators; and/or
one or more additives.

10. The deodorizing, anti-irritating, bactericidal, and launderable coating solution for brassieres of claim 9, wherein a ratio of 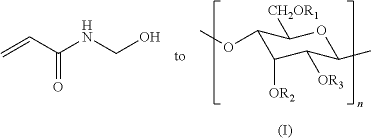
is 0.7:1 to 1.6:1 by weight.

11. The deodorizing, anti-irritating, bactericidal, and launderable coating solution for brassieres of claim 9, wherein the compound of formula I includes
CH₂CH₂SO₃⁻Na⁺ (II)
and a total number of
CH₂CH₂SO₃⁻Na⁺ (II)
is 2.

12. The deodorizing, anti-irritating, bactericidal, and launderable coating solution for brassieres of claim 9, wherein the compound of formula I includes
CH2CH2SO3 Na+ (II) and a total number of
CH2CH2SO3 Na+ (II) is 4.

13. The deodorizing, anti-irritating, bactericidal, and launderable coating solution for brassieres of claim 9, wherein the initiator is selected from one or more of 2,2′-azobis [2-(2-imidazolin-2-yl) propane] disulfate dihydrate, 2,2′-azobis [2-(2-imidazolin-2-yl)propane] dihydrochloride, 2,2′-azobis (2-methylpropionamidine) dihydrochloride, 2,2′-azobis [2-(2-imidazolin-2-yl) propane], 2,2′-azobis [n-(2-carboxyethyl)-2-methylpropionamidine] n-hydrate, 2,2′-azobis [2-methyl-n-(2-hydroxyethyl)propionamide], or 4,4′-azobis (4-cyanovaleric acid).

14. The deodorizing, anti-irritating, bactericidal, and launderable coating solution for brassieres of claim 9, wherein the one or more additives is selected from acrylic acid, 2-(dimethylamino) ethyl acrylate, 3-(trimethoxysilyl)propyl acrylate, glycidyl acrylate, methacrylic acid, 2-acrylamido-2-methylpropanesulfonic acid, N-[3-(dimethylamino)-propyl] acrylamide, N-[2-(dimethylamino)-ethyl] acrylamide, N-(3-dimethylaminopropyl)-methacrylamide.

15. The deodorizing, anti-irritating, bactericidal, and launderable coating solution for brassieres of claim 9, wherein the coating solution further comprises a metal salt.

16. The deodorizing, anti-irritating, bactericidal, and launderable coating solution for brassieres of claim 15, wherein the metal salt is selected from one or more of zinc acrylate, sodium acrylate, potassium acrylate, zinc methacrylate, 2-acrylamido-2-methylpropanesulfonate sodium salt or 3-sulfopropyl acrylate potassium salt.

17. A deodorizing, anti-irritating, bactericidal, and launderable brassiere comprising:
a brassiere including one or more fabric portions, the fabric portions including one or more of nylon, polyester, cotton, and polyurethane, including fabric blends of one or more of nylon, polyester, cotton, and polyurethane; the brassiere having a deodorizing and anti-irritating coating formed thereon, the deodorizing and anti-irritating coating including:
a deodorizing, bactericidal and fungicidal component of 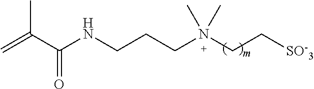
wherein m is 2, 3, 4, 5, 6, 7, 8, 9
and a deodorizing component that includes the following compound: 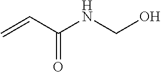
and a cyclodextrin compound according to the following formula (I) 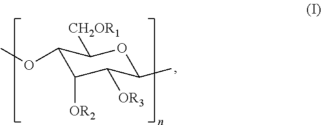
wherein n is 6, 7, 8; R1, R2, and R3 are jointly or independently selected from H or
CH₂CH₂SO₃⁻Na⁺ (II)
wherein the total number of groups represented by formula (II) can be 2 or 4;
and
one or more crosslinkers;
and
one or more initiators; and/or
one or more additives;
wherein, a deodorizing performance against isovaleric acid is at least 95 percent when measured using an ISO 17299-3 test method; and
wherein, an antibacterial performance against Staphylococcus aureus and Klebsiella pneumonia is at least 99.9 percent when measured using an AATCC 100-2019 test method and wherein an antibacterial performance against Staphylococcus aureus and Klebsiella pneumonia is at least 99.9 percent when measured using an AATCC 100-2019 test method after the brassiere has undergone 30 laundry cycles.

18. (canceled)

19. The deodorizing, anti-irritating, bactericidal, and launderable brassiere according to claim 17, wherein a ratio of 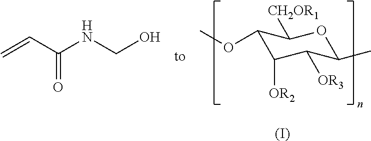
is 0.7:1 to 1.6:1 by weight.

20. The deodorizing, anti-irritating, bactericidal, and launderable brassiere according to claim 17, wherein the compound of formula I includes
CH₂CH₂SO₃⁻Na⁺ (II)
and a total number of
CH₂CH₂SO₃⁻Na⁺ (II)
is 2.

21. The deodorizing, anti-irritating, bactericidal, and launderable brassiere according to claim 17, wherein the compound of formula I includes
CH₂CH₂SO₃⁻Na⁺ (II)
and a total number of
CH₂CH₂SO₃⁻Na⁺ (II)
is 4.

22. The deodorizing, anti-irritating, bactericidal, and launderable brassiere according to claim 17, wherein the coating has a weight ranging from approximately 10.3 to approximately 70.9 g/m².

23. The deodorizing, anti-irritating, bactericidal, and launderable brassiere according to claim 17, wherein the coating is silver-free.
